(19) United States
(12) Patent Application Publication
(10) Pub. No.: US 2010/0280303 A1
(43) Pub. Date: Nov. 4, 2010

(54) DEVICES AND METHODS FOR TREATING MAGNETIC POISONING AND/OR MAGNETICALLY INDUCED ROULEAUX

(76) Inventor: Dan L. Dietz, Houston, TX (US)

Correspondence Address: Dan L. Dietz, 10234 Emerald Drive, Houston, TX, 77074, US

(21) Appl. No.: 12/433,566

(22) Filed: Apr. 30, 2009

(51) Int. Cl. A61N 2/00; A61M 1/10

(52) U.S. Cl. 600/12; 600/15; 600/16; 604/151; 600/18

(57) ABSTRACT

A medical device including a Rouleaux degausser that directs a degaussing magnetic field to a patient's blood flow. The degaussing magnetic field reduces magnetically-induced Rouleaux and/or Red Blood Cell aggregation. The device also has a power source for supplying power to the Rouleaux degausser.

### TECHNICAL FIELD

The present disclosure relates generally to devices and methods for treating blood and, more particularly, to devices and methods for treating Rouleaux.

### BACKGROUND

Rouleaux is a blood condition wherein red-blood cells (RBC) stick together in a configuration similar to a stack of coins. Typically, four to ten RBC stack together, although larger or smaller stacks are possible. In some instances, Rouleaux can slow the circulation of blood within a lumen of the human body. This slowing can result in limited physiological function, and in some situations medical problems. In addition, Rouleaux is sometimes accompanied by cellular aggregation, i.e., the random or substantially random “clumping” or adherence of RBC and/or multiple distinct Rouleaux formations.

Various medical devices utilize magnets and other components that produce magnetic fields. Such medical devices can include imaging devices, internal and external pumps, pacemakers, and sensors. Patients having exposure to such devices are therefore often exposed to high levels of magnetic fields. These magnetic fields can be caused by electromagnetic radiation, permanent and electric magnets, or other sources.

After significant research, the inventor has determined that Rouleaux formation can be influenced by magnetic fields. In particular, the inventor has determined that certain static magnetic fields can magnetize individual RBC's, and thereby induce Rouleaux. Although this process is not precisely understood, it is believed that a static magnetic field can induce magnetization of the iron containing hemoglobin proteins contained within individual RBC's. As a result, such fields may impart a net dipole moment to individual RBC's. Such induced magnetization is believed to be an underlying cause of Rouleaux formation associated with devices having magnetic components.

Degaussing is a process of decreasing or eliminating an unwanted magnetic field. Due to magnetic hysteresis, it is generally not possible to reduce a magnetic field to zero. As such, degaussing typically results in a small magnetic field remaining in the degaussed material.

Degaussing has traditionally been used to erase data stored in magnetic media. Magnetic media include small regions containing magnetic domains. These domains have a magnetic alignment that can be altered by an external magnetic field. Data is stored in magnetic media, such as hard drives, by making these domains change their magnetic alignment to be in the direction of an applied magnetic field. Degaussing aims to randomly orient the magnetic domains in such media, thereby rendering previously recorded data unrecoverable.

Degaussing may be achieved in several ways. For example, AC degaussing applies an alternating magnetic field/signal that decreases in amplitude over time from an initial high value. Alternatively, DC degaussing involves applying a unidirectional field to scramble previously aligned magnetic domains.

Some prior devices have attempted to provide a therapeutic benefit through the application of magnetic fields. For example, U.S. Pat. No. 6,461,288 (“the '288 patent”) to Holcomb discloses a device for altering the charge distribution on cellular membranes via the application of a static magnetic field. The '288 patent alleges that various disorders, including pain and cardiac dysfunction, may be treated with the static magnetic field produced by the device.

The device disclosed in European Patent No. 0 995 463 B1 (“the '463 patent”) to Wolf generates a pulsed electromagnetic field to influence human physiology. The pulsed electromagnetic field includes a plurality of individual pulses having a frequency between 1 and 1,000 Hz and an amplitude governed by a specific algorithm.

While the devices disclosed by the '288 and '463 patents may be useful for treating some human ailments, both devices generally fail to adequately treat certain types of Rouleaux. In particular, it is believed that these devices aim to disrupt chemical or adhesive bonds that are through to occur between cellular membranes in so-called “naturally occurring” Rouleaux. However, these devices are generally not adapted to address “magnetically-induced” Rouleaux, i.e., that which is believed to form as the result of exposure to certain magnetic fields.

Concern about the complications associated with magnetically-induced Rouleaux lead the inventor, after significant experimentation, to develop the devices and methods disclosed herein.

Accordingly, one aspect of the present disclosure describes devices and methods to treat magnetically-induced Rouleaux. In some embodiments, these devices and methods employ a degaussing magnetic field to eliminate or reduce abnormal magnetization of RBC's, which is believed to be an underlying cause of magnetically-induced Rouleaux.

### SUMMARY

Described herein are various devices, systems, and methods for treating magnetically induced Rouleaux. In one aspect, a medical device having a Rouleaux degausser and a power source is disclosed. The Rouleaux degausser directs a degaussing magnetic field at a patient's blood flow, wherein the degaussing magnetic field reduces or eliminates magnetically-induced Rouleaux and/or Red Blood Cell (RBC) aggregation. The power source supplies power to the Rouleaux degausser.

As used herein, the term “magnetic field” includes static and electromagnetic fields, unless otherwise expressly stated.

Another aspect of the current disclosure is directed to an implantable medical device (IMD). The IMD can include at least one magnet having an associated magnetic field, wherein the field interacts with a patient's blood flow to aggregate Red Blood Cells (RBC). The IMD further includes a Rouleaux shield located about the IMD to at least partially attenuate said magnetic field exposed to the patient's blood flow.

Another aspect of the current disclosure is directed to Ventricular Assist Device (VAD). The VAD can include an impeller configured to rotate and cause blood flow, wherein the impeller can include at least one vane. The VAD can further include a stator configured to apply a magnetic field to rotate the impeller, wherein said at least one magnet is positioned substantially within the impeller so as to reduce Red Blood Cell (RBC) aggregation relative to a VAD having a magnet positioned within a vane.

Yet another aspect of the current disclosure is directed to a method for treating a patient. The method can include applying a degaussing magnetic field to a patient's blood flow, wherein the degaussing magnetic field at least partially degausses Red Blood Cells (RBC).

It is to be understood that both the foregoing general description and the following detailed description are exemplary and are not restrictive of the present disclosure, as claimed. In addition, structures and features described with respect to one embodiment can similarly be applied to other embodiments.

### BRIEF DESCRIPTION OF THE DRAWINGS

The accompanying drawings, which are incorporated in and constitute a part of this specification, provide illustrative embodiments of the present disclosure and, together with the description, serve to explain the disclosure's principles.

FIG. 1 illustrates a schematic representation of one exemplary embodiment of the present disclosure.

FIG. 2 illustrates a side-view of a patient wearing one exemplary embodiment of the present disclosure.

FIG. 3 illustrates a cross-sectional side-view of one embodiment of a Ventricular Assist Device described herein.

FIG. 4 illustrates another cross-sectional top-view of one embodiment of a Ventricular Assist Device described herein.

FIG. 5 illustrates another cross-sectional top-view of one embodiment of a Ventricular Assist Device described herein.

### DETAILED DESCRIPTION

Disclosed herein are devices and methods for treating magnetically-induced Rouleaux. As outlined above, Rouleaux can be induced by exposing blood to a magnetic field, in particular a static magnetic field. Such fields are believed to induce magnetization of the iron containing hemoglobin in the Red Blood Cells (RBC). The magnetic polarization resulting from this induced magnetization is believed to contribute to the formation of stacked aggregates (i.e., Rouleaux). These stacked RBC can hinder blood circulation, limiting physiological function, and possibly initiating or exacerbating pathological conditions.

The devices and methods of the present disclosure treat magnetically-induced Rouleaux. In some embodiments, the disclosed devices includes a Rouleaux degausser that applies a degaussing magnetic filed, such as an electromagnetic field, to reduce or eliminate magnetization of RBC. If such degaussing signal is applied to aggregated RBC, it may reduce or eliminate such aggregation by reducing or eliminating any magnetic attraction between individual RBC. Such devices may also include a Rouleaux shield that reduces exposure of the patient's blood flow to magnetic fields. Also described are methods for treating magnetically-induced Rouleaux via application of the disclosed devices.

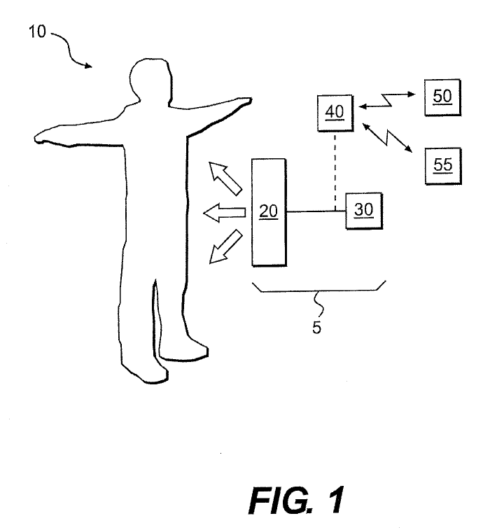

FIG. 1 provides a schematic representation of one exemplary embodiment of a medical device 5 for treating Rouleaux. Device 5 includes a Rouleaux degausser 20 and a power source 30. Power source 30 provides electrical energy to degausser 20. For example, power source 30 can include a battery, mains power, fuel cell, or other source of electrical energy. In addition, power source 30 may be rechargeable.

Rouleaux degausser 20 applies a magnetic signal to the blood flow of a patient 10. For example, degausser 20 can include a coil (e.g., an induction coil), an electromagnetic device and/or material, or a similar device/material capable of applying a degaussing magnetic field. As discussed below in more detail, degausser 20 can be implanted within patient 10 or located about patient 10.

Degausser 20 is designed to apply a degaussing magnetic field, wherein the field acts to de-magnetize RBC and/or induce magnetization on RBC that approximates the natural magnetic moment of a typical RBC. In some embodiments, the application of the degaussing signal may reduce or eliminate RBC aggregation by reducing or eliminating abnormal magnetic attraction between individual RBC.

To affect the magnetization of RBC, the degaussing magnetic field must have sufficient field strength to overcome the coercivity of magnetic domains of the RBC. “Coercivity” (generally reported in Oersted) is generally known in the art as a relative measure of the intensity of an applied magnetic field that is necessary to reduce magnetization of a material to zero, once the magnetization of that material has been driven to saturation. Provided the degaussing signal has sufficient field strength, its application to RBC may be sufficient to orient the magnetic domains of the RBC in a random or substantially random fashion, thereby reducing or eliminating the magnetization of the RBC. Alternatively, application of the degaussing signal can be controlled so as to re-magnetize RBC in a way that substantially emulates the impact of the electromagnetic fields produced naturally within the human body. In either case, the application of the degaussing signal may reduce abnormal magnetization of RBC and thus, reducing or eliminate abnormal magnetic interaction between individual RBC that is believed to be an underlying cause of magnetically-induced Rouleaux and/or cellular aggregation.

Reducing magnetically-induced Rouleaux and/or cellular aggregation can be achieved by applying various degaussing magnetic fields, including degaussing static and electromagnetic fields. For example, degaussing can be achieved by applying an electromagnetic field having a field intensity large enough to affect the magnetic properties of RBC. In some embodiments, the degaussing signal field strength may range from 400 milli-Gauss (mG) to 1,200 milli-Gauss (mG), or more.

Lower or higher intensity fields may also be used, depending on the size of the patient and the relative distance between the source of the degaussing signal to a patient's blood flow. Thus, for example, the degaussing signal's magnetic field strength can range from greater than about 0.004, 1, 1.2, 10, 50, 100, 1000, 2500, 5000, or 10,000 Gauss or more.

As examples of devices that may be suitably used as Degausser 20, non-limiting mention is made of degaussers commonly used to erase or scramble information stored on magnetic media, such as the Geneva PF-211 and PF-215 degaussers, which have degaussing electromagnetic field strengths of approximately 2300 and 2800 gauss, respectively. Of course, one of ordinary skill would understand that other devices producing a degaussing signal of sufficient field strength may also be used as Degausser 20.

In another non-limiting embodiment, magnetically-induced Rouleaux may be reduced or eliminated by the application of a magnetic field (e.g., a static or electromagnetic field) having a specific polarity. For example, an device (e.g., an implantable medical device) may contain components that produce a magnetic field having a certain polarity (e.g., north or south). Application of a static or electromagnetic magnetic field of opposite polarity could be applied to least partially cancel the magnetic field produced by the device in a particular spatial region about the source of magnetic field. As a result, the magnetic field intensity within such spatial region may not be of sufficient intensity to induce magnetization of RBC, thereby reducing or eliminating an underlying cause of magnetically-induced Rouleaux and/or cellular aggregation.

In another non-limiting embodiment of the present disclosure, a pulsed electromagnetic field may be applied to reduce or eliminate the impact of a static magnetic field on RBC. For example, a electromagnetic field of opposite polarity to the static magnetic field could be applied periodically or thereabouts. The periodicity of the electromagnetic field may, for example, be controlled so as to coincide (or approximately coincide) with the periodicity of a magnetic field applied by an implantable device. In addition, the orientation of the pulsed electromagnetic field may be controlled such that it is opposite the orientation of the magnetic field produced by the implantable device. By exercising such control, the pulsed electromagnetic field could serve to at least partially cancel the magnetic field produced by the implantable device, while minimizing patient exposure to the degaussing signal.

Alternatively, the degaussing signal could be applied out-of-phase (or substantially out of phase) with the magnetic field produced by an implantable device. Such degaussing could scramble the magnetic domains of RBC during periods of quiescent implantable device activity.

As a non-limiting example of the application of an out-of-phase degaussing signal, it is known that certain ventricular assist devices (“VAD” or “VADs”) employ an impeller having a magnetic element embedded within one or more vanes. Rotation of the impeller is caused via the interaction of the magnetic field produced by the embedded magnet (e.g, a static magnetic field) with a magnetic field produced by a coil disposed in proximity to the impeller (e.g., an electromagnetic field). The magnetic field associated with either the embedded magnet or the coil may be of sufficient intensity to induce magnetization of RBC and thus, lead to magnetically-induced Rouleaux and/or cellular aggregation.

To address this issue, the rotation of the VAD impeller could be adjusted to coincide with the application of a magnetic field (e.g., a pulsed electromagnetic field) from the associated coil. As such, the coil would have “active” periods (i.e., wherein the coil is actively producing a magnetic field) and “resting” periods (i.e., wherein the coil is not actively producing a magnetic field). The degaussing signal discussed herein (e.g., a pulsed electromagnetic field) may be applied during the resting periods of the coil, so as to reduce or eliminate the effect of the coil and/or embedded magnet fields on the magnetization of the RBC. Alternatively, the degaussing signal could be controlled such that the RBC's exhibit magnetization that is the same or substantially the same as that produced by natural body processes.

In other instances, a frequency, waveform, amplitude, or other parameters associated with the degaussing magnetic signal may be applied or varied so as to reduce Rouleaux and/or cellular aggregation. For example, parameters associated with the degaussing signal may be varied to account for variation in patient weight, age, or other patient-specific factors. These variations arise because magnetic field strength decreases exponentially with distance from the magnetic source. As such, a degaussing signal may require modification depending upon the type or location of degausser 20 used to treat patient 10. For example, a degausser configured for placement about the torso of patient 10 may require the use of a degaussing signal having stronger field strength than a degausser configured for placement about the arm or leg of patient 10.

In some embodiments, part or all of device 5 may be implantable. For example, degausser 20 could be located about a VAD, as described briefly above and in more detail below. In other examples, degausser 20 could include an implantable cuff configured for partial placement about a blood vessel or organ, such as the heart or lungs.

Power source 30 may also be located within or external to patient 10. For example, while degausser 20 may be implanted adjacent to an implantable device, power source 30 could be worn outside the body of patient 10. Power source 30 could be in the form of a holster battery worn about the waist of patient 10. This configuration may include an electrical connection between degausser 20 and power source 30, via wires or via wireless transcutaneous energy transfer.

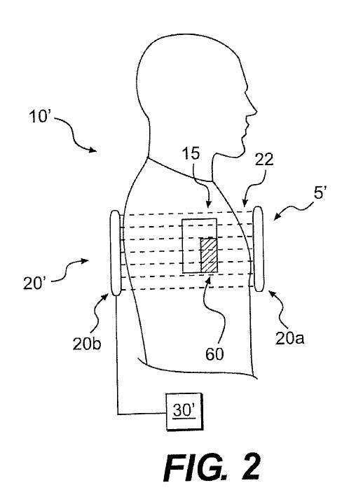

In some embodiments, all or part of device 5 may be configured for external placement about the body of patient 10. For example, device 5 or components thereof, may be placed on or about an arm, leg, or torso of patient 10. Specifically, degausser 20 may include a cuff configured for placement about a limb of patient 10. Also, as shown in FIG. 2, degausser 20 may include a medallion, vest, or other object worn about patient 10.

FIG. 2 illustrates a side-view of patient 10′ wearing one exemplary embodiment of a device 5′. As shown in FIG. 2, device 5′ include an external power source 30′ and an external degausser 20′. External degausser 20′ includes a ventral medallion 20a and a dorsal medallion 20b. Medallions 20a, 20b are positioned about the torso of patient 10′ and configured to apply a degaussing signal 22. In some embodiments, medallions 20a and/or 20b may be positioned in close proximity to a source of a static magnetic filed originating in close proximity to the body.

As outlined above, degaussing signal 22 affects the magnetic properties of RBC of patient 10′. Specifically, degaussing signal 22 is a magnetic field transmitted by ventral source 20a and dorsal source 20b. In some embodiments, medallion 20a may be a ventral source of a degaussing signal, and medallion 20b may be a dorsal source of such field. Alternatively, medallion 20a and medallion 20b may be used as independent ventral and/or dorsal sources.

As shown, signal 22 is designed to reduce magnetically-induced Rouleaux formed within patient 10′ by an implantable medical device 60. As explained below, implantable device 60 can include any number of medical devices whose operation includes applying magnetic fields to the blood of patient 10′. As shown in FIG. 2, implantable device 60 is a pump configured to operate in conjunction with a an organ 15 of patient 10′, such as a heart.

As shown in FIG. 1, device 5 can include a processor 40. Processor 40 can control one or more operations associated with device 5, and may be powered by power source 30 or another source of electrical energy. Various commercially available microprocessors can be adapted or programmed to perform one or more functions of processor 40. A memory, a secondary storage device, a secondary or parallel processor, may operate with processor 40. Other components associated with processor 40 could include power supply circuitry, signal conditioning circuitry, graphical display circuitry, or user interface circuitry.

Processor 40 may embody a single microprocessor or multiple microprocessors configured to control the degaussing magnetic field applied to the patient's blood. Processor 40 can provide continuous or intermittent control to regulate the degaussing magnetic field applied by device 5. For example, processor 40 could transmit a signal to power source 30 to activate degausser 20 to transmit a degaussing signal, e.g., a degaussing static or electromagnetic field.

Also, processor 40 could monitor a magnetic field associated with device 5. Based on reception of such a signal, processor 40 may regulate the degaussing magnetic field transmitted by device 5. Processor 40 may further transmit a signal to one or more components of device 5, such as, for example, degausser 20 or power source 30.

Processor 40 may receive a signal representative of a magnetic field associated with device 5. For example, a signal representative of a magnetic field associated with degausser 20 can be received by processor 40. In operation, a sensor (not shown) may be located and output a signal associated with an operation of device 5. For example, a magnetic field sensor (not shown) could be located within patient 10 or adjacent to degausser 20. The sensor could include any suitable type of magnetic field sensor and may measure magnetic field amplitude, frequency, flux, or orientation. Such a signal can be sent continuously, intermittently, or when requested by processor 40.

In some situations, processor 40 can perform a calculation to convert a received signal into any suitable representative value associated with a magnetic field. For example, the signal may include a current or voltage reading received from a sensor mounted within or about patient 10. Further, processor 40 could compare this signal to another signal received from a separate sensor located on another part of patient 10 to determine a relative value of magnetic field, or other representation of magnetic field.

Any suitable signal processing or algorithm to convert an input signal into a value associated with a magnetic field could be included in processor 40. For example, processor 40 could use other sensory inputs as a substitute for the magnetic signal. Such inputs may be associated with various patient parameters, such as, for example, blood temperature, blood viscosity, patient feedback, or blood appearance as observed under a microscope. Processor 40 may receive and analyze such input to derive a representative magnetic field value, or alter an operation of device 5. For example, if processor 40 received a signal from a sensor indicating a high magnetic field, processor 40 could then transmit a signal to decrease the degaussing magnetic field transmitted by degausser 20.

One or more signals could be transmitted, based on control by processor 40, to various components associated with of device 5. Processor 40 could control transmission of a signal to a programmer 50, wherein programmer 50 could provide a user interface (not shown) to permit reprogramming of device 5. Thus, programmer 50 could enable a physician or nurse to increase or decrease the intensity of a magnetic field applied to patient 10. Further, programmer 50 could be linked to a network and communication with processor 40 could occur via the internet.

In other aspects, programmer 50 could include a graphical display (not shown). Such a display could be configured to display one or more parameters associated with device 5 or patient 10. For example, blood pressure, magnetic field strength, heart rate, and other data could be displayed using programmer 50.

One or more signals transmitted from device 5 or programmer 50 may be received by processor 40. For example, processor 40 could receive a signal representative of a power level associated with power source 30. A low power level from power source 30 may trigger an alarm or a signal to be transmitted to programmer 50.

Processor 40 could output a signal to control one or more operations of device 5, such as, for example, the magnetic field output by device 5. In particular, processor 40 can control a magnetic field transmitted by degausser 20. For example, processor 40 could transmit at least one signal to degausser 20, power source 30, or other subsystems of device 5 to maintain a degaussing magnetic field within a desired range. In particular, processor 40 may control the flow of electrical power to degausser 20 from power source 30, or control degausser 20 directly, to vary the degaussing magnetic field transmitted by degausser 20.

In some embodiments, device 5 may operate in conjunction with a second medical device 55. Second device 55 could include any implantable or external medical device configured to apply a magnetic field to the blood of patient 10. For example, second device 55 could include a heart pump, a drug-delivery pump (e.g., a Heparin-delivery pump), a dialysis pump, or other type of pump.

Second device 55 could also include a Magnetic Resonance Imaging (MRI) device or other device producing a strong static and/or electromagnetic field, such as, for example, a nuclear magnetic resonance (NMR) device. As explained previously, such magnetic fields may cause Rouleaux by inducing abnormal magnetization of RBC. Due to the intensity of the magnetic fields associated with these devices, magnetically-induced Rouleaux and/or cellular aggregation may occur even if a patient is exposed to such fields for a short period of time.

Processor 40 may operate with second device 55 to control one or more operations of device 5 or second device 55. For example, processor 40 could communicate with second device 55 to control the degaussing magnetic field transmitted to patient 10. Specifically, a degaussing magnetic field could be altered during operation of second device 55 so that the degaussing magnetic field does not interfere with an operation of second device 55.

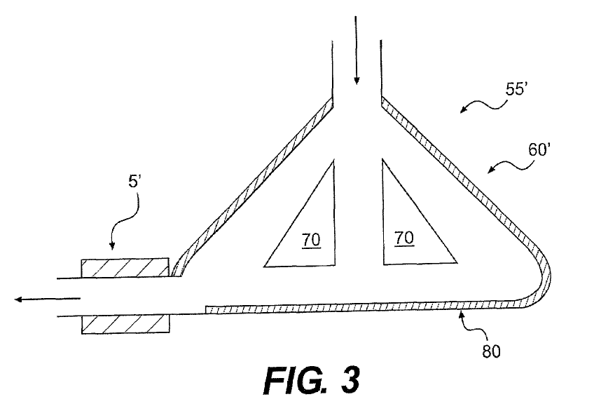

By way of example, FIG. 3 shows a medical device 5′ associated with a second medical device 55′. As shown in FIG. 3, second device 55′ is an implantable medical device 60′, specifically a Ventricular Assist Device (VAD). VAD 60′ can include an axial flow or centrifugal flow blood pump configured to provide temporary, mechanical ventricular support.

VAD 60′ includes an impeller 70, wherein impeller 70 rotates to cause blood flow. Rotation of impeller 70 is controlled by applying a magnetic field (e.g., an electromagnetic field) to magnets (not shown) located within impeller 70. The magnetic field causes the magnets to move relative to the applied field, thereby causing rotation of impeller 70, and blood flow. The applied magnetic field can, under certain conditions, cause Rouleaux via magnetically-induced RBC aggregation.

As shown in FIG. 3, blood enters at the top of VAD 60′ and exits out of the bottom left. As outlined above, device 5′ is configured to apply a degaussing signal, such as an electromagnetic field to the blood within VAD 60′ to reduce RBC aggregation. Although device 5′ is shown positioned at the blood flow exit of VAD 60′, device 5′ could also be positioned at the blood intake of VAD 60′.

Device 5′ could be located upstream or downstream of VAD 60′ such that the degaussing signal produced by device 5′ does not affect the operation of VAD 60′. However, volume or design constraints may require that device 5′ and VAD 60′ are sufficiently close so that the operation of one affects the operation of the other. Because VAD 60′ may be affected by the degaussing signal provided by device 5′, the operations of device 5′ and VAD 60′ may be inter-dependent.

As previously described, device 5′ includes a processor (not shown in FIG. 3) that can control an operation of device 5′ or second device 55′. Because the operations of devices 5′, 55′ can be dependent upon one another, suitable control of either or both devices 5′, 55′ is possible. For example, device 5′ could apply a degaussing signal when VAD 60 is operating in a safe mode. Such a safe mode could include stopping VAD 60, or running VAD 60 at a slower or higher speed. In another example, device 5′ could alter an operation of VAD 60 to permit the safe application of a degaussing signal by device 5′.

In another example, the operation of device 5′ could be directly dependent upon the operation of VAD 60′. Specifically, VAD 60′ could apply a train of alternating pulses to spin impeller 70. As described above, a degaussing signal, such as a pulsed electromagnetic field (degaussing signal pulse), may be applied out-of-phase or in-phase with a magnetic field used to drive impeller 70 (impeller pulse). Specifically, a degaussing signal pulse may be applied while no impeller pulse is applied. Such a degaussing signal would demagnetize RBC in between pulses applied to spin impeller 70. In addition, such a degaussing signal may also reduce Rouleaux without significantly affecting the operation of VAD 60′.

In yet another embodiment, a processor (not shown) within device 5+ could be incorporating within the control circuitry of VAD 60′. Such a processor could be configured to modify an operation of VAD 60′ or the magnetic field applied by VAD 60′ to at least partially reduce Rouleaux. As a result, a separate degausser may not be required to degauss a patient's blood flow. For example, VAD 60′ could be operated intermittently or the magnetic field orientation within VAD 60′ could be alternated or varied, so as to limit exposure of a patients blood to magnetic fields sufficient to cause magnetically-induced Rouleaux.

In another aspect, the implantable devices disclosed herein may include a Rouleaux shield. For example, VAD 60′ can include a Rouleaux shield 80. Blood flowing through organs located about VAD 60′ can be exposed to its magnetic field. Shield 80 can be constructed and positioned so as to reduce magnetic field exposure to organs located adjacent to VAD 60′.

Shield 80 can include any suitable magnetic shielding material or structure. Non-limiting examples of suitable materials include those having high magnetic permeability and/or dielectric constant, such as, for example, ferrites, NiFe based alloys such as Mu-metal (a range of known Nickel, Iron, Copper, Molybdenum alloys), Permalloy (a range of known nickel-iron alloys, e.g., Ni₈₁Fe₁₉), Magnifer (a range of known nickel-iron-molybdenum alloys that further include manganese and optionally other additive elements), and cobalt-iron alloys such as Permendur (a range of known cobalt-iron-vanadium alloys, e.g., Co₄₈₋₅₀FeV₀₋₂). Coatings of these materials can attenuate a magnetic signal produced by second device 55′, thereby reducing the magnetic field interacting with the RBC of patient 10. Shield 80 can also operate via blocking of reflection of an incident magnetic field. For example, shield 80 could be configured in the form of a Faraday Cage manufactured from any number of well known conductive materials, such as metals, conductive polymers, conductive composites, etc.

To maintain biocompatibility of an implantable device, Rouleaux shield 80 may be disposed within a known biocompatible material, or sandwiched between layers of such biocompatible materials. For example, Rouleaux shield 80 may be disposed between two layers of the well known Ti-6Al-4V alloy that is commonly used to form implanted medical devices. Of course, other biocompatible materials (e.g., biocompatible metals, alloys and/or polymeric materials) may be used to surround Rouleaux shield 80, so as to maintain biocompatibility of the implanted device.

Shield 80 could be located at least partially about, or at least partially within, VAD 60′ or a component of VAD 60′. For example, as shown in FIG. 3, shield 80 extends substantially about the outer perimeter of device 55′.

In some embodiments, VAD 60′ could include shield 80 without device 5′. Such a passive VAD 60′ could operate to shield the surrounding organs and tissue from unwanted magnetic fields produced by VAD 60′ or any other type of second device 55′. For example, an MRI system could at least partially include shield 80 to reduce magnetic field exposure to part of a patient's blood flow.

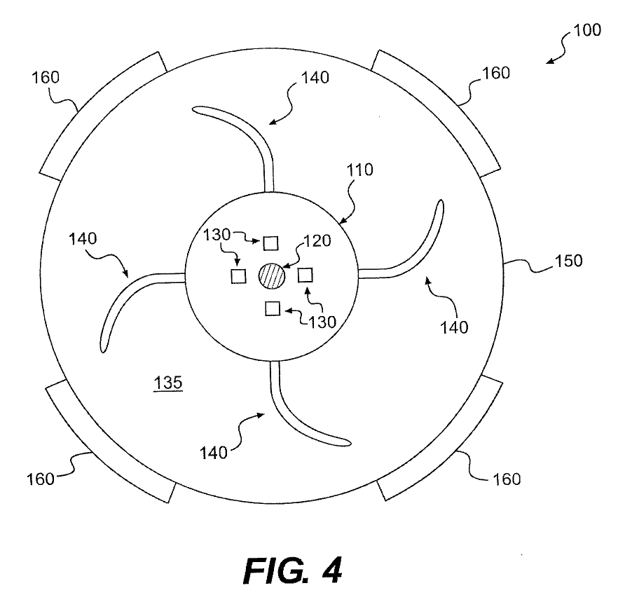

FIG. 4 illustrates another cross-sectional view of one embodiment of a VAD 100. In this embodiment, VAD 100 includes an impeller 110 and a shaft 120. In some instances, shaft 120 may not be required. For example, if impeller 110 utilizes a hydrodynamic bearing or other bearing not requiring physical connection between impeller 110 and a VAD housing 150.

VAD 100 may include one or more electric coils 160 positioned about housing 150. Coils 160 may be controlled by a processor (not shown) and supplied with electrical power by a power source (not shown). Magnetic fields produced by coils 160 (e.g., electromagnetic fields) interact with one or more magnets 130 (e.g, static magnets) to cause impeller 110 to rotate and cause blood flow. Of course, coils 160 may also be positioned within housing 150 (not shown), or disposed between housing 150 a secondary housing manufactured from biologically compatible material (also not shown), so as to limit or prevent contact of coils 160 with the surrounding biological materials.

As shown, four magnets 130 are shown within impeller 110. Magnets have traditionally been located within or about the periphery of impeller vanes, shown as feature 140 in FIG. 4. Here, by contrast, magnets 130 are located close to the center axis of impeller 110 and generally away from blood flowing through a chamber 135 of VAD 100. Such positioning places the magnetic field produced by magnets 130 away from the blood within chamber 135, thus reducing the exposure of RBC to the field produced by magnets 130.

Blood may be further positioned away from the magnetic field produced by magnets by one or more large vanes 140 located about impeller 110. In particular, vanes 140 may be larger than traditional designs, permitting enhanced blood flow with lower magnetic fields. In addition, impeller 110 may have a diameter larger than traditional impellers. Again, such a configuration can reduce the exposure of blood to magnetic fields produced within VAD 100.

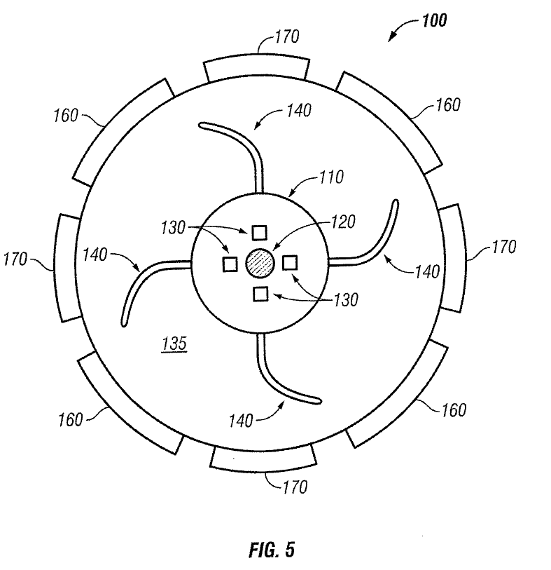

FIG. 5 illustrates another non-limiting embodiment of the present disclosure, wherein four degaussers 170 are interposed radially between two or more coils 160. Each of degaussers 170 may be configured to apply a degaussing signal, such as a pulsed electromagnetic field (degaussing signal pulse) sufficient to prevent, limit, and/or negate any induced RBC magnetization resulting from the magnetic fields produced by coils 160. As described substantially above, for example, degaussers 170 may be configured to apply degaussing pulses that are out-of-phase or in-phase with the magnetic field (e.g, a pulsed electromagnetic field or intermittent static magnetic field) produced by coils 160 to drive impellers 140 (impeller pulse). For example, degaussing signal pulse(s) may be applied while no impeller pulse(s) is/are applied. Such a degaussing signal may be configured demagnetize RBC in between pulses applied to spin impeller 140. In addition, such a degaussing signal may also reduce Rouleaux without significantly affecting the operation of VAD 100.

Further, degaussers 170 may be individually or collectively controlled by a processor. For example, a processor may direct each degausser 170 to apply a degaussing signal at the same or different time as another degausser 170, so as to obtain a desired degaussing effect.

While FIG. 5 illustrates an embodiment wherein four degaussers 170 are employed, more or less degaussers may be used. For example, at least one, two, three, four, or more degaussers 170 may be disposed on or about coil 160. Similar to FIG. 4, coils 160 and degaussers 170 may also be disposed within housing 150 (not labeled in FIG. 5, or disposed between housing 150 and an secondary housing manufactured from biologically compatible material (also not shown), so as to limit or prevent contact of coils 160 and degaussers 170 with the surrounding biological materials.

Other than in the operating examples, or where otherwise indicated, all numbers expressing quantities of ingredients, reaction conditions, and so forth in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches. Also, where a range is given, even if the term “between” is used, the ranges defined include the stated endpoints.

Notwithstanding the numerical ranges and parameters setting forth the broad scope of the invention as approximations, the numerical values set forth in the specific examples are reported as precisely as possible. Any numerical value, however, inherently contains certain errors necessarily resulting from the standard deviation found in its respective testing measurement.

The examples that follow serve to illustrate the invention without, however, being limiting in nature. The characteristics and advantages of this disclosure will now be demonstrated by the following non-limiting examples. Comparative example 2 is reported for purposes of comparison as an example using the known technique. In the following examples, the “emptying test” comprised weighing the liquid released by manually compressing the container.

For all patents, applications, or other reference cited herein, it should be understood that such documents are incorporated by reference in their entirety for all purposes, as well as for any specifically recited proposition. Where any conflict exists between a document incorporated by reference and the present application, this application will dominate.

### EXAMPLES

A study was performed on a single male subject following the topical application of a 2,000 mG static neodymium magnet to the lower-left clavicle region. Topical application of such a static magnet has been shown in prior studies to induce Roulleaux and/or aggregation of living peripheral blood tissue as seen with biological (phase contrast) microscopy.

Testing was performed to determine the efficacy of several different electromagnetic signal generators on the formation of magnetically induced Roulleaux and/or cellular aggregation. These field generators were held in close proximity to the static magnet, and the efficacy of such application was determined visually using phase contrast microscopy.

Equipment

All of the following studies were performed using a BioMedx Biological Imaging System, including a phase contrast microscope, a trinocular head, a Lumina fiber optic supply, and a camera. Image recording was performed through a Canopus Digital Video Converter, model no. ADVC110. A magnetic pen supplied by Dietz Designs was used as the source of the 2000 mG static magnetic field.

Very weak electromagnetic field generation was as produced by a Bemer 3000 Mat on level ten, as supplied by Bemer USA, LLC. Weak electromagnetic field generation was as produced by a Bemer 3000 Intensive applicator on level 10, as supplied by Bemer USA, LLC. Strong electromagnetic field generation was as produced by the Geneva PF-211 and PF-215 tape video/audio tape demagnetizers/degaussers. Certain specifications of the studied electromagnetic field generators are provided in Table 1 below.

TABLE 1

| | | | | Bemer |
|---|---|---|---|---|
| | Geneva | Geneva | Bemer 3000 | Intensive |
| | PF-211 | PF-215 | Mat | Applicator |
| Voltage | 120 VAC | 120 VAC | 120 VAC | 120 VAC |
| Frequency | 60 Hz | 60 Hz | Y = k(x) * X^{a} * e^{sin}(x^{b}), | |
| | | | k(x) 1, a = b = 3 | |
| Power | 125 W | 125 W | 6 W | 6 W |
| Current | 6.0 A | 9.5 A | 0.4 A | 0.4 A |
| Signal | 125, 0.0, 3.5 | 158, 0.0, 1.2 | 35 μT | 100 μt |
| strength at | | | | |
| 19.5 in. | | | | |
| (x, y, z) | | | | |

Methodology

Magnetic effects were studied on a single male subject during a one-day period. Peripheral blood samples were collected from alternate different fingers on different hands. Before and after samples were taken with near-field (close) static magnetic application along the left collar line. The wait time between individual tests was approximately 20 minutes.

Multiple sets of data and visual measurements were obtained and evaluated subjectively. Video clips of the observed visual phenomena were recorded.

Magnetic Field Measurements

Because the test location was in an unshielded office and the effect being studied was statically and electromagnetically induced, measuring was performed to identify and verify the ambient magnetic field strength in the tested degaussing signal generators. Electro-magnetic ambient fields in all three domains were measured around the test room, at the microscope stage and power supply, near the AC adaptor strip and at a distance of 19.5 inches from the two audio/video tape demagnetizers. Large magnetic fields were found at the AC transformer strip. However, no sufficiently strong fields were measured over a ten-minute interval during setup. None of the participants in the testing had any magnetic materials on their person, nor had any been exposed to strong static magnetic or electromagnetic fields for at least two hours prior to the testing. The peak ambient room measurements are provided in Table 2 below.

TABLE 2
Baseline Measurements

| | Location of Ambient | |
|---|---|---|
| | Magnetic Field | Measurement |
| | Measurements | (x, y, z) mG |
| Microscope | x, y, z field at microscope | 1.21, 0.0, 0.62 |
| Lumina Fiber Optic | x, y, z, field at fiber optic | 1.18, 3.16, 3.77 |
| | x, y, z, field at magnetic | 1.37, 0.0, 0.77 |
| | North | |
| | x, y, z, field at magnetic | 1.21, 0.02, 1.42 |
| | East | |
| | x, y, z, field at magnetic | 1.14, 0.63, 0.71 |
| | South | |
| | x, y, z, field at magnetic | 1.04, 0.05, 0.64 |
| | West | |
| Largest AC transformer | x, y, z, field at transformer | 192.4, 0.0, 1.14 |
| PF-215 | x, y, z, field at 19.5 inches | 158, 0, 1.2 |
| PF-211 | x, y, z, field at 19.5 inches | 125, 0, 3.5 |

In Vitro Baseline: No Applied Static or Electromagnetic Field

Prior to application of a static or electromagnetic field, a blood sample was taken from a single finger-stick of the subject. This sample was applied to a microscope slide (Slide 1), and was left in a separate room and observed in ten-minute intervals as a control. Slide 1 maintained vital blood integrity over the recorded forty-minute duration of observation. Slight (˜15%) Rouleaux formation was observed at the forty-minute point.

A 2,000 mG static magnetic was applied at the subject's left collar line, and samples were drawn from the subject's right hand at two minute intervals for ten minutes, and observed. The samples were drawn from difference fingers to avoid temporary localized inflammatory effects.

TABLE 3
Application of Static Magnet Alone

| Slide/Sample | Time (minutes) | Hand | Finger |
|---|---|---|---|
| 2 | 2 | Right | Index |
| 3 | 4 | Right | Middle |
| 4 | 6 | Right | Ring |
| 5 | 8 | Right | Pinky |
| 6 | 10 | Right | Thumb |

Observation confirmed that the application of the static magnetic field produced nearly immediate and complete Rouleaux. Over the ten minute application period, the level of Rouleaux decreased to around 10%. Although not precisely understood at this time, the inventor believes that the reduction in Rouleaux is attributable to some “compensation” mechanism within the body. In similar prior testing, approximately forty minutes were required for this effect to occur.

Simultaneous Application of Static Magnet and Bemer Intensive Applicator

In an additional study, the 2000 mG static magnet was applied to the subject's left collar line. At the same time, the Bemer Intensive Applicator was held directly on top of the static magnet, and was set at power level 10. Samples were drawn from the subject's left hand at two minute intervals for ten minutes, and observed. The samples were drawn from difference fingers to avoid temporary localized inflammatory effects.

TABLE 4
Simultaneous Application of Static Magnet and Bemer intensive
Applicator at Level 10

| Slide/Sample | Time (minutes) | Hand | Finger |
|---|---|---|---|
| 7 | 2 | Left | Index |
| 8 | 4 | Left | Middle |
| 9 | 6 | Left | Ring |
| 10 | 8 | Left | Pinky |
| 11 | 10 | Left | Thumb |

Total or near total cancellation of the static magnetically induced Rouleaux effect was observed, provided the Bemer Intensive Applicator remained on and applied directly over the 2000 mG static magnet. The control unit of the Bemer applicator shut off the electromagnetic signal at the eight minute mark. Two minutes later approximately 20% Rouleax was observed, indicating that the low-level electromagnetic signal may need to remain constantly on in order to “cancel” magnetically induced Rouleaux.

Simultaneous Application of Static Magnet and the Geneva Model 211 and 215 Degaussers

In a further study, the 2000 mG static magnet was applied to the subject's left collar line. At the same time, either the Geneva Model PF-211 degausser or the Model PF-215 degausser was held directly on top of the magnetic pen for one full minute (the suggested operational duration of the degausser) and then turned off. In the PF-211 samples, both the degausser and the static magnetic field were removed at the one minute interval. In the PF-215 samples, the degausser was removed the one minute interval, but the static magnet was left in place. Samples were taken from the subject at the 1, 2, 4 and 6 minute intervals for the PF-211 application, and at 1, 2, 4, 6, 8, 10, 12, 14, 16, and 18 minute intervals for the PF-215 application.

TABLE 5
Simultaneous Application of Static Magnet and Geneva Degausser

| | | | Time | | |
|---|---|---|---|---|---|
| Slide/Sample | PF-211 | PF-215 | (minutes) | Hand | Finger |
| 12 | x | | 1 | Right | Index |
| 13 | x | | 2 | Right | Middle |
| 14 | x | | 4 | Right | Ring |
| 15 | x | | 6 | Right | Pinky |
| 16 | | x | 1 | Right | Index |
| 17 | | x | 2 | Right | Middle |
| 18 | | x | 4 | Right | Ring |
| 19 | | x | 6 | Right | Pinky |
| 20 | | x | 8 | Right | Thumb |
| 21 | | x | 10 | Left | Index |
| 22 | | x | 12 | Left | Middle |
| 23 | | x | 14 | Left | Ring |
| 24 | | x | 16 | Left | Pinky |
| 25 | | x | 18 | Left | Thumb |

In the samples exposed to the PF-211, near total cancellation of the magnetically induced Rouleaux effect was observed. Approximately 5% Rouleaux formed after the six-minute test period. The samples exposed to the PF-215 also showed near total cancellation of the magnetically induced Rouleaux effect in sample 17. In sample 18, approximately 90% Rouleaux was observed. The Rouleaux then steadily decreased over the next ten minutes. Over the remaining four minutes, Rouleaux formation began increasing and at the end of the test period was approximately 30%.

Simultaneous Application of Static Magnet and the Bemer 3000 Mat

In another study, the 2000 mG static magnet was applied to the subject's left collar line. At the same time, the subject laid prone on the Bemer 3000 mat, which was activated at power level 10. Samples were drawn from the subject's right hand at two minute intervals for ten minutes, and observed.

TABLE 6
Simultaneous Application of Static Magnet and the Bemer 3000 Mat

| Slide/Sample | Time (minutes) | Hand | Finger |
|---|---|---|---|
| 26 | 2 | Right | Index |
| 27 | 4 | Right | Middle |
| 28 | 6 | Right | Ring |
| 29 | 8 | Right | Pinky |
| 30 | 10 | Right | Thumb |

Observation showed that magnetically induced Rouleaux formation occurred as usual (i.e., as expected without the application of the mat) at the two-minute and four-minute intervals. The results suggest that the Bemer Mat has insufficient field strength to prevent magnetically induced Rouleaux formation, even though it applies a field across the entire surface of the body. It was also observed that the normally expected “compensation” effect did not occur in the same way, suggesting that the Bemer Mat has some, though uncharacterized, effect.

While various embodiments have been illustrated and described above, it will be appreciated that various changes can be made therein without departing from the spirit and scope of the present disclosure. It is, therefore, intended that the scope of the present disclosure be determined from the following claims and equivalents thereof.

What is claimed is:

1. A medical device, comprising:
a Rouleaux degausser for directing a degaussing magnetic field at a patient's blood flow, wherein the degaussing magnetic field reduces static magnetically-induced Rouleaux and/or Red Blood Cell (RBC) aggregation; and
a power source for supplying power to the Rouleaux degausser.

2. The medical device of claim 1, wherein the Rouleaux degausser is located about the patient's body.

3. The medical device of claim 2, wherein the Rouleaux degausser comprises at least one of a cuff, a medallion, and a vest.

4. The medical device of claim 1, wherein the Rouleaux degausser is implantable.

5. The medical device of claim 1, wherein the Rouleaux degausser further comprises a coil configured to apply the degaussing magnetic field.

6. The medical device of claim 1, further comprising a processor configured to transmit a signal to control the degaussing magnetic field applied to the patient's blood flow by the Rouleaux degausser.

7. The medical device of claim 6, wherein the processor is further configured to operate with a second medical device, said second device includes a magnetic field source that is sufficient to magnetically-induce Rouleaux or RBC aggregation when disposed in proximity to a patient's blood flow.

8. The medical device of claim 7, wherein the second medical device comprises a pump.

9. The medical device of claim 8, wherein the pump is selected from the group consisting of a heart pump, a drug-delivery pump, and a dialysis pump.

10. The medical device of claim 7, wherein the Rouleaux degausser is configured to apply a degaussing magnetic field having an intensity greater than the coercivity of the magnetic domains of the RBC.

11. The medical device of claim 6, wherein the processor is further configured to operate with a programmer configured to reprogram an operation of the Rouleaux degausser.

12. An implantable medical device (IMD), comprising:
at least one source of a magnetic field, wherein said magnetic field interacts with a patient's blood flow to magnetically-induce at least one of Rouleaux and cellular aggregation, and
a Rouleaux shield located about the IMD to at least partially attenuate said magnetic field exposed to the patient's blood flow.

13. The IMD of claim 12, wherein the Rouleaux shield attenuates said magnetic field, thereby reducing or eliminating at least one of magnetically-induced Rouleaux and cellular aggregation in said blood flow.

14. The IMD of claim 13, wherein the Rouleaux shield comprises a coating material selected from the group consisting of ferrite, Mu-metal, CO-NETIC, Permendur, Permalloy, and Magnifer alloys.

15. The IMD of claim 12, wherein said IMD comprises at least one of a heart pump and a drug-delivery pump.

16. The IMD of claim 12, wherein the IMD further comprises a Rouleaux degausser having a degaussing magnetic field directed to a patient's blood flow, wherein the degaussing electromagnetic field reduces magnetically-induced Rouleaux and/or cellular aggregation.

17. A Ventricular Assist Device (VAD), comprising:
an impeller configured to rotate and cause blood flow, wherein the impeller comprises at least one vane; and
a stator coil configured to apply a magnetic field to rotate the impeller, wherein said at least one magnet is positioned substantially within the impeller so as to reduce Red Blood Cell (RBC) aggregation relative to a VAD having a magnet positioned within a vane.

18. A method for treating a patient, comprising:
applying a degaussing electromagnetic field to a patient's blood flow, wherein the degaussing magnetic field at least partially degausses Red Blood Cells (RBC) aggregated via magnetic-induction.

19. The method of claim 18, further including applying a magnetic field to a patient's blood flow to aggregate RBC.

20. The method of claim 18, further including modifying the degaussing magnetic field.
